(19) United States
(12) Patent Application Publication
(10) Pub. No.: US 2026/0284245 A1
(43) Pub. Date: Sep. 24, 2026

(54) APPARATUS FOR HEATING AND DISINFECTING TOWELS

(71) Applicant: Yao Yin, Nanning City (CN)

(72) Inventor: Yao Yin, Nanning City (CN)

(21) Appl. No.: 19/680,925

(22) Filed: May 18, 2026

(30) Foreign Application Priority Data
Feb. 6, 2026 (CN) 202620179824.8

(51) Int. Cl. A61L 2/08; A47K 10/06; A61L 103/50; C02F 1/32

(52) U.S. Cl. CPC A61L 2/08; A47K 10/06; C02F 1/325; A61L 2103/50; C02F 2201/326; C02F 2303/04

(57) ABSTRACT

The invention discloses an apparatus for heating and disinfecting towels, comprising a mounting bracket, a storage bin, a water storage tank, a workbench, a pushing assembly, a forming assembly, a water pump, a heater, a disinfection lamp and a controller. The storage bin and water storage tank are mounted on the mounting bracket, and the workbench is arranged below the storage bin. Towels are stacked vertically inside the storage bin and discharged from the bottom outlet to the loading area. The pushing assembly delivers a single towel to the working area. The heater is connected to the water storage tank via a pipeline, and the water pump conveys heated water to the forming assembly. The disinfection lamp sterilizes the water source. This instant heating device reduces energy consumption, prevents towel mildew and jamming by single-piece feeding, and owns compact structure and excellent sanitary performance.

### CROSS-REFERENCE TO RELATED APPLICATIONS

This application claims priority to Chinese Patent Application No. 202620179824.8 filed on Feb. 6, 2026, which is hereby incorporated by reference in its entirety.

### TECHNICAL FIELD

The present invention relates to the technical field of towel processing equipment, and in particular, to an apparatus for heating and disinfecting towels.

### BACKGROUND

Heated towels have the functions of cleaning and hot compression to relieve fatigue, and meet modern peoples demands for health and comfort in daily life. Therefore, they are widely applied in households, hotels, beauty salons and other scenarios. Traditional towel heating and disinfection devices need to pour hot water into the water storage tank or integrally heat the cold water stored in the tank, which leads to long waiting time and severe energy waste. Meanwhile, such devices only support single water temperature regulation, making it difficult to adapt to differentiated demands in different seasons and regions. At the same time, most existing devices adopt a structure design of spraying water first and then conveying towels. Towels will expand after water absorption and are prone to jamming. In addition, moisture tends to migrate to spare towels, easily causing mildew and deterioration of the spare towels.

### SUMMARY

The present invention aims to provide a towel heating and disinfecting apparatus, so as to solve the problems existing in the above background art.

To achieve the foregoing objective, the present invention provides the following technical solution;

A towel heating and disinfecting apparatus includes a mounting bracket, a storage bin, and a water storage portion for storing tap water to provide a water source. The water storage portion and the storage bin are both mounted on the mounting bracket. A workbench is disposed below the storage bin and mounted on the mounting bracket;

Towels are stacked along the height direction inside the storage bin, and a discharge outlet is disposed at one side of the bottom of the storage bin. One end of the workbench below the discharge outlet is defined as a loading area, and the other end is defined as a working area. Towels in the storage bin are placed on the loading area. A pushing assembly for pushing a single towel from the loading area to the working area is disposed on the side of the storage bin opposite to the discharge outlet;

A processing assembly is disposed on the workbench and located in the working area. A water pump and a heater for instantaneously heating tap water delivered from the water storage portion to a preset temperature are disposed at the bottom of the mounting bracket. A water inlet end of the heater is connected to the water storage portion via a pipeline, and the water pump is respectively connected to a water outlet end of the heater and the processing assembly via pipelines;

A disinfection lamp is disposed on the water storage tank to sterilize the tap water contained therein.

A controller is further disposed on the mounting bracket.

According to a preferred embodiment, the water storage portion comprises a water storage tank. A mounting frame is disposed on the mounting bracket; the water storage tank is mounted on the mounting frame. A water outlet pipe is disposed at the bottom of the water storage tank. The water outlet pipe is connected to the heater through a pipeline, and a filter element is disposed inside the water outlet pipe.

According to a preferred embodiment, the disinfection lamp is disposed at the top of the water storage tank, and the irradiation coverage of the disinfection lamp faces the inner cavity of the water storage tank to disinfect the water source inside the tank.

According to a preferred embodiment, the pushing assembly comprises two support frames. The two support frames are symmetrically disposed on the workbench and arranged on one side of the storage bin. Opposite inner sides of the two support frames are each provided with a mounting sleeve, and opening end faces of the two mounting sleeves are fitted together to define a mounting cavity;

A rotating sleeve and a coil spring are disposed in the mounting cavity. The rotating sleeve is rotatably connected to one of the support frames; a limit rod is disposed on the other support frame, the limit rod penetrates through the rotating sleeve, and the coil spring is sleeved on the limit rod;

One end of the coil spring is fixed to the limit rod, and the other end is fixed to the inner side of the rotating sleeve.

According to a preferred embodiment, the pushing assembly further comprises a pushing block and a steel push coil. A pushing groove is formed on one side of the storage bin corresponding to the discharge outlet; a storage groove is formed on the support frame, and the pushing block is disposed at the opening of the pushing groove and inside the storage groove;

A limit track is disposed on the inner side of the support frame, and an outlet of the limit track is arranged on one side of the storage groove. One end of the steel push coil is wound around the rotating sleeve, and the other end penetrates through the limit track and is connected to the pushing block Extension and retraction of the steel push coil drives the pushing block to move through the pushing groove, so as to push the bottommost towel in the storage bin out of the discharge outlet to the working area.

According to a preferred embodiment, a motor is disposed on one of the support frames. A pushing gear is mounted at a shaft end of the motor. A plurality of clamping grooves are defined on the pushing steel coil. Gear teeth of the pushing gear engage with the clamping grooves to form a meshing structure between the pushing gear and the pushing steel coil.

According to a preferred embodiment, a water outlet groove is defined on the workbench and located in the working area. A sealing member is disposed at the bottom of the workbench, and the sealing member and the workbench jointly enclose a water outlet cavity. A water inlet pipe is disposed at the bottom of the sealing member and connected to the water pump;

A plurality of water outlet holes are arranged in the water outlet groove, and the water outlet holes are in communication with the water outlet cavity.

According to a preferred embodiment, the workbench is further disposed with a first positioning sensor for detecting the position of a towel, and the first positioning sensor is disposed in the working area.

According to a preferred embodiment, a second positioning sensor for detecting the remaining quantity of towels in the storage bin is further disposed on one side of the storage bin, and the second positioning sensor is adjacent to the discharge outlet.

Compared with the prior art, the present invention has the following beneficial effects:
1. By adopting the structural design of the water storage tank and the instant heating heater, the present application does not need to inject hot water into the water storage tank or integrally heat the cold water inside the water storage tank. The heater realizes instant heating only during water delivery, which greatly shortens the waiting time for users and avoids energy waste caused by overall heating and heat preservation. The heating temperature can be set in cooperation with the controller, so as to effectively meet differentiated water temperature requirements in different seasons and regions, With the functional logic of “pushing first and spraying water later”, the pushing assembly pushes a single towel from the storage bin to the working area via the loading area, and then the processing assembly performs water spraying. The present application thoroughly solves the problem that towels are prone to jamming during pushing after absorbing water and expanding in conventional devices, and prevents mildew and deterioration of spare towels in the storage bin due to moisture migration, thus ensuring the cleanliness and sanitation of the spare towels.
2. The disinfection lamp installed on the water storage tank can continuously sterilize and disinfect the stored tap water, solving the problem that the water storage tank of traditional devices is prone to bacterial growth due to long-term water storage, and improving the hygiene of hot towel use; the large-capacity storage bin, combined with the single-piece pushing design of the pushing component, reduces the need for users to frequently add towels. Together with the integrated layout of the mounting bracket, the device is compact in structure, convenient to install, and suitable for use in various scenarios.
3. The water storage part adopts a mounting structure of a water storage tank combined with a mounting bracket, which not only ensures the stability of water storage but also facilitates later disassembly and maintenance. The connection method between the water outlet pipe and the pipeline of the heater ensures the tightness and smoothness of water flow transmission; the disinfection lamp is installed on the top of the water storage tank with its irradiation range facing the inside of the tank, realizing full-coverage sterilization of the water source and further improving the hygiene guarantee of water use; the pushing assembly achieves accurate and stable towel pushing movement through the coordinated cooperation of two sets of support frames, a rotating sleeve, a coil spring, a pushing steel coil and a pushing block, combined with a motor-driven gear meshing structure. The elastic reset function of the coil spring enables the pushing block to return quickly, preparing for the next pushing operation, and the limit track ensures the movement trajectory of the pushing steel coil, avoiding pushing deviation.
4. The water outlet groove in the working area, combined with the through design of multiple groups of water outlet holes and the water outlet cavity, enables hot water to be evenly sprayed on the towel, ensuring that the towel absorbs water sufficiently and is heated uniformly; the arrangement of the sealing member effectively prevents hot water leakage; the first positioning sensor can accurately detect the position of the towel in the working area to ensure the accurate triggering of the water spraying action; the second positioning sensor can real-time monitor the stock of towels in the storage bin, facilitating timely reminding the user to replenish materials; the above two cooperate with the controller to realize the automatic coordinated operation of the device, further improving the convenience of use and operational stability.

### BRIEF DESCRIPTION OF DRAWINGS

FIG. 1 is a structural schematic diagram of an apparatus for heating and disinfecting towels according to the present invention after assembly;

FIG. 2 is a structural schematic diagram of the apparatus for heating and disinfecting towels according to the present invention after disassembly;

FIG. 3 is a structural schematic diagram of a pushing assembly of the apparatus for heating and disinfecting towels according to the present invention;

FIG. 4 is a structural schematic diagram of the pushing assembly of the apparatus for heating and disinfecting towels according to the present invention after disassembly;

FIG. 5 is a structural schematic diagram of a heater and a water pump of the apparatus for heating and disinfecting towels according to the present invention.

Reference Numerals; 11. Mounting bracket; 21. Storage bin; 22. Discharge outlet; 23. Pushing groove; 24. Second positioning sensor; 31. Workbench; 32. Water pump; 33. Heater; 34. Disinfection lamp; 35. Controller; 36. Water outlet groove; 41. Water storage tank; 42. Mounting frame; 43. Water outlet pipe; 44. Sealing member; 45. Water inlet pipe; 46. First positioning sensor; 501. Support frame; 502. Mounting sleeve; 503. Rotating sleeve; 504. Coil spring; 505. Limit rod; 506. Pushing block; 507. Pushing steel coil; 508. Storage groove; 509. Limit track; 510. Motor; 511. Pushing gear; 512. Clamping groove.

### DESCRIPTION OF EMBODIMENTS

The technical solutions in the embodiments of the present invention will be described clearly and completely below with reference to the accompanying drawings in the embodiments of the present invention. Obviously, the described embodiments are only part of the embodiments of the present invention, not all of them. Based on the embodiments of the present invention, all other embodiments obtained by ordinary skilled persons in the art without creative work shall fall within the protection scope of the present invention.

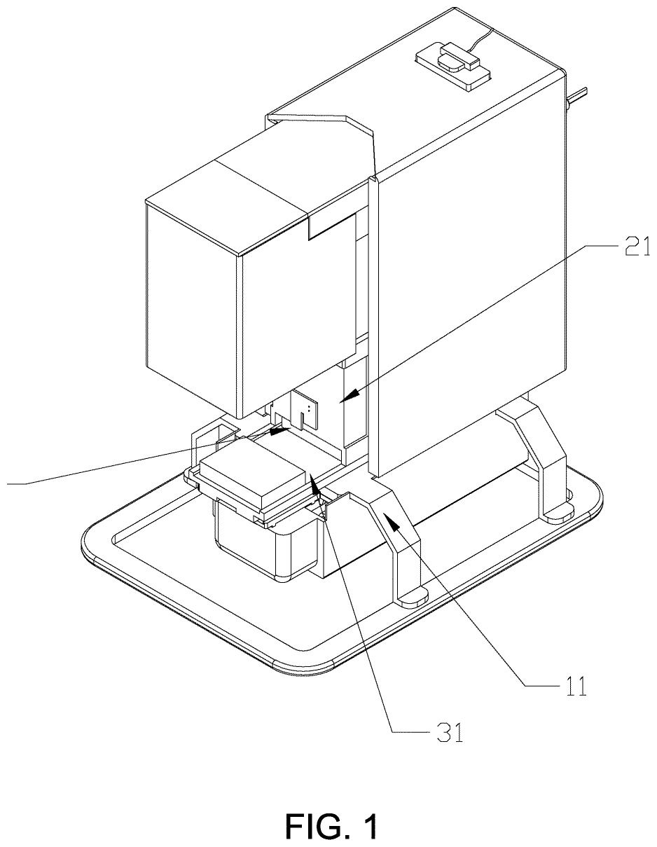

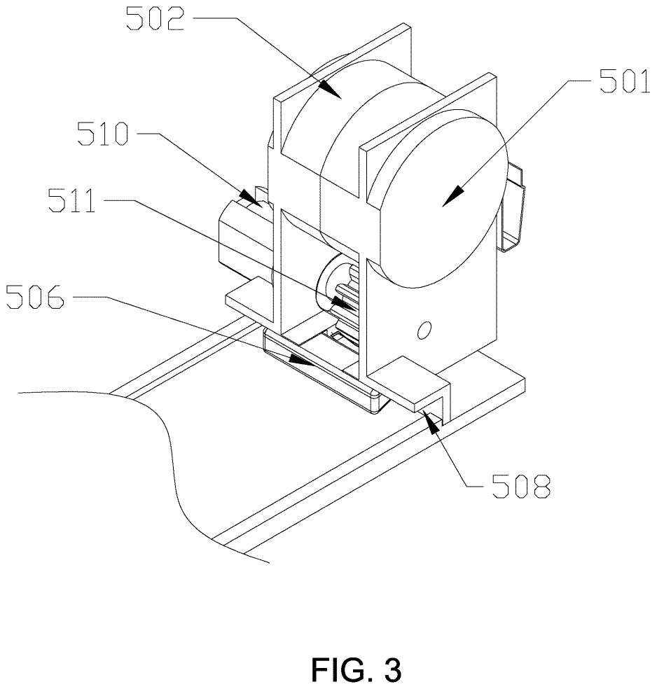

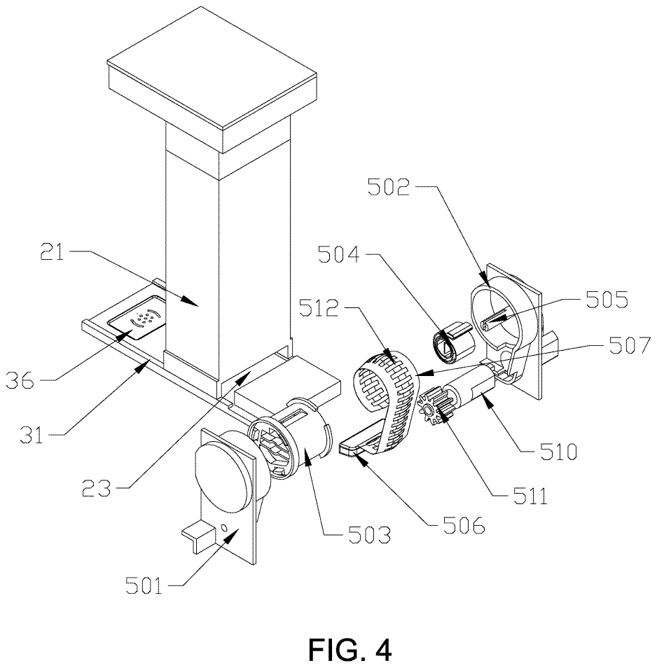

Please refer to FIG. 1 to FIG. 5. The present invention provides a towel heating and disinfection apparatus, comprising a mounting bracket 11, a storage bin 21, and a water storage tank for storing tap water and supplying a stable water source for preparing hot towels. The water storage tank and the storage bin 21 are both fixedly mounted on the mounting bracket 11. The mounting bracket 11 supports the overall structure and ensures the positional stability of each assembled component, and avoids displacement or shaking during operation;

A workbench 31 is disposed below the storage bin 21, and the workbench 31 is fixedly connected to the mounting bracket 11 by bolts to form a layered mounting structure. Towels are stacked neatly along the height direction of the storage bin 21. The storage bin 21 provides a closed storage space for the towels and reduces contamination of the towels by external dust. A discharge outlet 22 is disposed at one side of the bottom of the storage bin 21; the size of the discharge outlet 22 matches the thickness and width of a single towel to ensure the smooth passage of the towel. One end of the workbench 31 below the discharge outlet 22 is configured as a loading area, which receives a single towel falling from the storage bin 21 and provides initial support for the subsequent pushing action.

The other end of the workbench 31 is configured as a working area, which provides an operation platform for preparing hot towels. Towels in the storage bin 21 are placed on the loading area. A pushing assembly is disposed on the side of the storage bin 21 opposite to the discharge outlet 22. The pushing assembly can generate horizontal thrust to drive a single towel to move smoothly from the loading area to the working area, so as to realize orderly conveyance of towels. A processing assembly is disposed on the workbench 31 and located in the working area, and is configured to spray hot water onto the towels conveyed to the working area to complete hot towel preparation.

A water pump 32 and a heater 33 are disposed at the bottom of the mounting bracket 11. The heater 33 is configured to instantly heat tap water conveyed from the water storage tank to a preset temperature during water supply. The water inlet end of the heater 33 is hermetically connected to the water storage tank via a conduit to prevent water leakage during water delivery. The water pump 32 is connected via conduits to the water outlet end of the heater 33 and the processing assembly, respectively. The water pump 32 provides water conveying power to smoothly deliver the heated hot water to the processing assembly.

A disinfection lamp 34 is disposed on the water storage tank. The disinfection lamp 34 continuously irradiates the tap water stored in the water storage tank to destroy the bacterial structure in the water, achieve a sterilization and disinfection effect, and ensure the hygiene of the water source. A controller 35 is further disposed on the mounting bracket 11. The controller 35 is electrically connected to the water pump 32, the heater 33, the disinfection lamp 34, the pushing assembly and subsequent positioning sensors respectively. The controller receives signal feedback from each component, issues operation commands to each component, and coordinates the cooperative operation of all components.

The water storage part comprises a water storage tank 41 made of waterproof and corrosion-resistant materials, which can store tap water for a long time and is not prone to damage. A mounting frame 42 is disposed on the mounting bracket 11. The mounting frame 42 adopts an L-shaped structure. One end thereof is welded and fixed to the mounting bracket 11, and the other end is connected to the water storage tank 41 in a buckled manner. After the water storage tank 41 is mounted on the mounting frame 42, its position is kept fixed to prevent shaking, a water outlet pipe 43 is disposed at the bottom of the water storage tank 41. The water outlet pipe 43 is in communication with the interior of the water storage tank 41, and the pipe diameter of the water outlet pipe 43 matches that of the conduit. The water outlet pipe 43 is connected to the heater 33 via a conduit, such that tap water inside the water storage tank 41 can flow into the conduit through the water outlet pipe 43 and then be conveyed to the heater 33. Meanwhile, a filter element is disposed inside the water outlet pipe 43. The filter element adopts a fine filter screen structure, which can intercept impurities possibly mixed in tap water during storage or conveyance, and prevent impurities from entering the heater 33 or the processing assembly to cause pipeline blockage or adversely affect the hot water spraying effect.

The disinfection lamp 34 is mounted on the top of the water storage tank 41. The irradiation range of the disinfection lamp 34 faces the water inside the water storage tank 41, and the irradiation area can cover most of the interior of the water storage tank 41, ensuring that tap water at different positions inside the tank can be irradiated. Continuous light irradiation inhibits bacterial growth, reduces the quantity of harmful microorganisms in water, achieves comprehensive disinfection of the internal water source, and enables the water source ultimately used for preparing hot towels to meet hygiene standards.

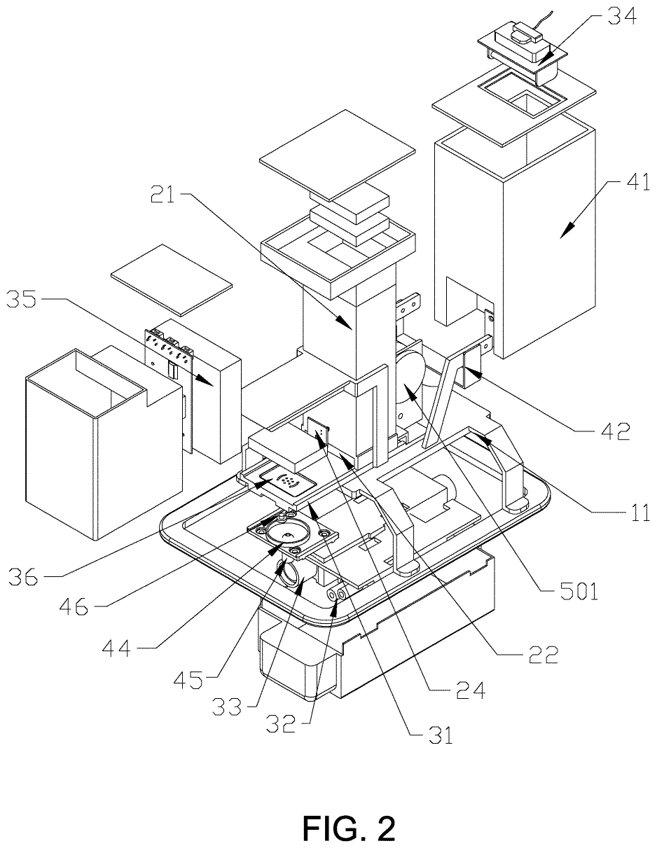

Please refer to FIG. 2 to FIG. 4. The pushing assembly includes two support frames 501. The two support frames 501 are symmetrically mounted on the workbench 31 and located on the same side of the storage bin 21. The support frames 501 are fixed to the workbench 31 with screws to serve as a mounting base for other components of the pushing assembly. Opposite inner sides of the two support frames 501 are each provided with a mounting sleeve 502. The mounting sleeve 502 and the support frame 501 are integrally formed. Opening end faces of the two mounting sleeves 502 abut each other to define a closed mounting cavity, which provides a protective space for internal components.

A rotating sleeve 503 and a coil spring 504 are disposed in the mounting cavity. The rotating sleeve 503 is rotatable about its own axis within the mounting cavity. The rotating sleeve 503 is rotatably connected to one of the support frames 501 via a bearing to reduce rotational friction and achieve smoother rotation. A limiting rod 505 is disposed on the other support frame 501. The limiting rod 505 is vertically fixed to the support frame 501 and extends through the rotating sleeve 503 to limit the rotational path of the rotating sleeve 503 and prevent positional deviation. The coil spring 504 is sleeved on the limiting rod 505 and has an elastic reset capability. One end of the coil spring 504 is retained in a positioning groove defined on the limiting rod 505 for fixation, and the other end is retained on an inner sidewall of the rotating sleeve 503. When the rotating sleeve 503 rotates, the coil spring 504 is torsionally deformed and stores energy. When the external force is removed, the coil spring 504 drives the rotating sleeve 503 to reversely rotate and return to its initial position.

The pushing assembly further includes a pushing block 506 and a pushing steel strip coil 507. A pushing groove 23 is defined on a side of the storage bin 21 corresponding to the discharge opening 22. A receiving groove 508 is defined on the support frame 501. The pushing block 506 is disposed at an opening of the pushing groove 23 and located inside the receiving groove 508. When not in operation, the pushing block 506 is received in the receiving groove 508 to avoid occupying extra space;

A limiting guide rail 509 is disposed on an inner side of the support frame 501. The limiting guide rail 509 is an elongated groove structure extending along a towel pushing direction. An outlet end of the limiting guide rail 509 is located on one side of the receiving groove 508 and corresponds to the position of the pushing block 506. The pushing steel strip coil 507 is made of high-strength steel with desirable toughness and structural strength. One end of the pushing steel strip coil 507 is wound around the rotating sleeve 503 and can be wound or unwound along with rotation of the rotating sleeve 503. The other end extends through the limiting guide rail 509. The limiting guide rail 509 guides the moving direction of the pushing steel strip coil 507 to prevent offset during movement. The other end of the pushing steel strip coil 507 is fixedly connected to the pushing block 506. When the pushing steel strip coil 507 is extended under driving of the rotating sleeve 503, it generates a pulling force to drive the pushing block 506 to pass through the pushing groove 23. After the pushing block 506 contacts the bottommost towel in the storage bin 21, it applies a pushing force to push the towel out of the discharge opening 22 and ultimately convey the towel to a working area.

A Motor 510 Is Mounted on One of the Support Frames 501. the Motor 510 Is Fixed to an outer side of the support frame 501 with bolts and serves as a power source for the pushing assembly. A pushing gear 511 is disposed at a shaft end of the motor 510. The pushing gear 511 is fixedly connected to an output shaft of the motor 510 and rotates synchronously therewith. A plurality of locking grooves 512 are defined on the pushing steel strip coil 507 and evenly distributed along a length direction thereof. Gear teeth of the pushing gear 511 are engaged with the locking grooves 512 to form a meshing structure between the pushing gear 511 and the pushing steel strip coil 507. When the motor 510 is activated, the pushing gear 511 rotates and drives the pushing steel strip coil 507 to wind or unwind via meshing between the gear teeth and the locking grooves, thereby controlling movement of the pushing block 506.

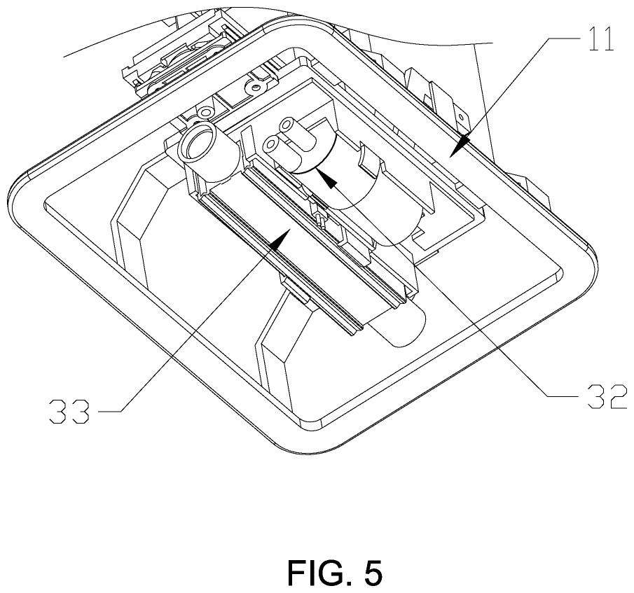

Please refer to FIG. 2 and FIG. 5. A water outlet groove 36 is defined on the workbench 31 and located in the working area, corresponding to a position of a towel conveyed to the working area. The water outlet groove 36 provides an accommodating space for hot water spraying and prevents hot water from flowing randomly over the workbench 31. A sealing member 44 is disposed at a bottom of the workbench 31. The sealing member 44 is made of rubber material with superior sealing performance and is fixedly connected to the bottom of the workbench 31 to form a closed water outlet cavity for temporarily storing hot water delivered by a water pump 32. A water inlet pipe 45 is disposed at a bottom of the sealing member 44 and communicates with the water outlet cavity. The water inlet pipe 45 is connected to the water pump 32 via a conduit, such that hot water delivered by the water pump 32 can flow into the water outlet cavity through the water inlet pipe 45. Multiple sets of water outlet channels are defined in the water outlet groove 36, evenly distributed at a bottom thereof and communicating with the water outlet cavity. Hot water in the water outlet cavity is uniformly sprayed upward through the plurality of water outlet channels, ensuring that the towel fully absorbs hot water and is heated uniformly.

The workbench 31 is further provided with a first positioning sensor 46. The first positioning sensor 46 is mounted in the working area adjacent to one side of the water outlet groove 36. The first positioning sensor 46 is configured to emit a detection signal. When a towel is pushed to a preset position in the working area, the towel blocks the detection signal of the first positioning sensor 46, which generates a trigger signal and transmits the trigger signal to a controller 35. Upon receiving the trigger signal, the controller 35 confirms that the towel is in place, and then controls the water pump 32 and a heater 33 to start operation. When a user removes the prepared hot towel, signal blocking is released. The first positioning sensor 46 feeds a signal back to the controller 35, and the controller 35 activates the pushing assembly to push out a subsequent towel to ensure continuous operation of the apparatus.

A second positioning sensor 24 is further disposed on one side of the storage bin 21. The second positioning sensor 24 is adjacent to the discharge opening 22, and its mounting height corresponds to a bottom position of stacked towels inside the storage bin 21. The second positioning sensor 24 determines whether towels remain in the storage bin 21 by detecting a distance between the sensor and the towels. When towels are present in the storage bin 21, the second positioning sensor 24 continuously transmits a material-present signal to the controller 35. When the quantity of towels in the storage bin 21 decreases to a preset value or is exhausted, the second positioning sensor 24 fails to detect any towel and transmits a material-shortage signal to the controller 35. The controller 35 then reminds a user to replenish towels into the storage bin 21 in a timely manner through indicator light flashing or voice prompt, thereby avoiding idle running of the apparatus.

The above descriptions are merely preferred exemplary embodiments of the present invention. The scope of protection of the present invention is not limited thereto. Any equivalent substitution or modification made by those skilled in the art without departing from the technical solution and inventive concept disclosed herein shall fall within the scope of protection of the present invention.

What is claimed is:

1. An apparatus for heating and disinfecting towels, comprising a mounting bracket (11), a storage bin (21), and a water storage assembly for storing tap water to provide a water source, wherein:
The water storage assembly and the storage bin (21) are both mounted on the mounting bracket (11); a workbench (31) is disposed below the storage bin (21) and mounted on the mounting bracket (11);
Towels are stacked along a height direction of the storage bin (21), and a discharge opening (22) is defined at one side of a bottom of the storage bin (21). One end of the workbench (31) below the discharge opening (22) is defined as a loading area, and the other end is defined as a working area. Towels from the storage bin (21) are placed on the loading area; a pushing assembly for pushing a single towel from the loading area to the working area is disposed on a side of the storage bin (21) opposite to the discharge opening (22);
A processing assembly is disposed on the workbench (31) and located in the working area; a water pump (32) and a heater (33) for instantly heating tap water delivered from the water storage assembly to a preset temperature are disposed at a bottom of the mounting bracket (11); a water inlet end of the heater (33) is connected to the water storage assembly via a conduit, and the water pump (32) is respectively connected to a water outlet end of the heater (33) and the processing assembly via conduits;
A disinfection lamp (34) for sterilizing tap water stored in the water storage assembly is disposed on the water storage assembly; a controller (35) is further disposed on the mounting bracket (11).

2. The apparatus for heating and disinfecting towels according to claim 1, wherein:
The water storage assembly includes a water storage tank (41); a mounting frame (42) is disposed on the mounting bracket (11), and the water storage tank (41) is mounted on the mounting frame (42); a water outlet pipe (43) is disposed at a bottom of the water storage tank (41); the water outlet pipe (43) is connected to the heater (33) via a conduit, and a filter cartridge is disposed inside the water outlet pipe (43).

3. The apparatus for heating and disinfecting towels according to claim 2, wherein, the disinfection lamp (34) is mounted on a top of the water storage tank (41), and an irradiation range of the disinfection lamp (34) faces an interior of the water storage tank (41) to realize disinfection and sterilization of an internal water source.

4. The apparatus for heating and disinfecting towels according to claim 1, wherein:
The pushing assembly comprises two support frames (501); the two support frames (501) are symmetrically mounted on the workbench (31) and located on one side of the storage bin (21); mounting sleeves (502) are disposed on opposite inner sides of the two support frames (501); opening end faces of the two mounting sleeves (502) abut each other to define a mounting cavity;
A rotating sleeve (503) and a coil spring (504) are disposed in the mounting cavity; the rotating sleeve (503) is rotatably connected to one of the support frames (501); a limiting rod (505) is disposed on the other support frame (501); the limiting rod (505) extends through the rotating sleeve (503), and the coil spring (504) is sleeved on the limiting rod (505);
Ane end of the coil spring (504) is retained on the limiting rod (505), and the other end is retained inside the rotating sleeve (503).

5. The apparatus for heating and disinfecting towels according to claim 4, wherein:
The pushing assembly further comprises a pushing block (506) and a pushing steel strip coil (507), a pushing groove (23) is defined on a side of the storage bin (21) corresponding to the discharge opening (22), a receiving groove (508) is defined on the support frame (501), the pushing block (506) is disposed at an opening of the pushing groove (23) and located inside the receiving groove (508);
A limiting guide rail (509) is disposed on an inner side of the support frame (501), and an outlet end of the limiting guide rail (509) is located on one side of the receiving groove (508), one end of the pushing steel strip coil (507) is wound around the rotating sleeve (503), and the other end extends through the limiting guide rail (509) and connected to the pushing block (506), extension of the pushing steel strip coil (507) drives the pushing block (506) to pass through the pushing groove (23), so as to push the bottommost towel in the storage bin (21) out of the discharge opening (22) to the working area.

6. The apparatus for heating and disinfecting towels according to claim 5, wherein:
A motor (510) is disposed on one of the support frames (501); a pushing gear (511) is disposed at a shaft end of the motor (510); a plurality of locking grooves (512) are defined on the pushing steel strip coil (507); gear teeth of the pushing gear (511) are engaged with the locking grooves (512) to form a meshing structure between the pushing gear (511) and the pushing steel strip coil (507).

7. The apparatus for heating and disinfecting towels according to claim 6, wherein:
a water outlet groove (36) is defined on the workbench (31) and located in the working area; a sealing member (44) is disposed at a bottom of the workbench (31); the sealing member (44) cooperates with the workbench (31) to form a water outlet cavity; a water inlet pipe (45) is disposed at a bottom of the sealing member and the water inlet pipe (45) is connected to the water pump (32);
A plurality of water outlet channels are defined in the water outlet groove (36), and the water outlet channels communicate with the water outlet cavity.

8. The apparatus for heating and disinfecting towels according to claim 6, wherein:
the workbench (31) is further provided with a first positioning sensor (46) for detecting a towel position, and the first positioning sensor (46) is mounted in the working area.

9. The apparatus for heating and disinfecting towels according to claim 1, wherein:
A second positioning sensor (24) for detecting towel stock inside the storage bin is further disposed on one side of the storage bin (21), and the second positioning sensor (24) is disposed adjacent to the discharge opening (22).
